(19) United States
(12) Patent Application Publication
(10) Pub. No.: US 2025/0032658 A1
(43) Pub. Date: Jan. 30, 2025

(54) SYSTEMS AND METHODS FOR NEUTRALIZATION OF AIRBORNE ALLERGENS WITH ORGANIC ACIDS

(71) Applicant: The Clorox Company, Oakland, CA (US)

(72) Inventors: Thomas Fahlen, Pleasanton, CA (US); Fatima Hussain, Pleasanton, CA (US); Alex Mayer, Pleasanton, CA (US); Yintao Sun, Pleasanton, CA (US); Shauna Flynn, Pleasanton, CA (US); Mark Pszczolkowski, Pleasanton, CA (US); Marjan Hashemi, Pleasanton, CA (US)

(21) Appl. No.: 18/782,421

(22) Filed: Jul. 24, 2024

Related U.S. Application Data
(60) Provisional application No. 63/515,677, filed on Jul. 26, 2023.

(51) Int. Cl. A61L 9/14; A61L 101/34; A61L 101/36; A61L 101/40

(52) U.S. Cl. CPC A61L 9/145; A61L 2101/34; A61L 2101/36; A61L 2101/40; A61L 2209/132; A61L 2209/135; A61L 2209/21

(57) ABSTRACT

A system includes an anti-allergen formulation and a device that generates droplets of the formulation to be delivered to an air environment. The aqueous formulation includes an organic acid having a vapor pressure between 0.001 Pa and 172 Pa and an aqueous solubility of at least 24.99 grams organic acid per 100 grams water, both at room temperature and standard pressure. The device can generate droplets of the formulation having a median particle size (Dv50) of no greater than 120 micrometers. The formulation delivered as droplets in an air environment can provide a statistically significant reduction, over a control, in one or more airborne allergens as measured by an enzyme linked immunoassay. In addition, the formulation delivered as droplets in the air environment can be effective to neutralize greater than 50% of one or more airborne allergens present in the air environment.

### CROSS-REFERENCE TO RELATED APPLICATION

The present application claims priority to U.S. Provisional Patent Application Ser. No. 63/515,677, filed Jul. 26, 2023, the disclosures of which is incorporated herein by reference in its entirety.

### FIELD

The present invention is directed toward effectively neutralizing airborne allergens utilizing formulations including organic acids. Methods and systems that provide effective delivery of the formulations in air via a variety of mechanisms including, without limitation, aerosols, misters, pump sprayers, HVLP sprayers, ULV foggers, thermal foggers, electrostatic sprayers, and/or ultrasonic dispensing devices.

### BACKGROUND

Certain allergens, particularly allergens that trigger allergies and asthma in individuals (e.g., pollens, pet dander, dust mites, mold spores, etc.), can be a problem in households and other enclosed environments. Certain products, e.g., HEPA and other types of filtration systems as well as anti-allergen sprays, can be used to help remove and/or neutralize allergens so as to reduce the impact on allergic reactions by individuals within such environments. While filtration systems can be helpful to remove airborne allergens, many anti-allergen sprays are typically configured for use in neutralizing allergens that collect on surfaces. It can be difficult, e.g., to provide a spray that provides an ingredient which is deemed safe for use and is further active to neutralize allergens in the air.

Accordingly, it would be advantageous to provide a formulation and system that facilitates safe and effective neutralization of airborne allergens.

### BRIEF SUMMARY

In accordance with embodiments described herein, a system comprises an anti-allergen formulation and a device that generates droplets of the formulation to be delivered to an air environment. The aqueous formulation comprises an organic acid having a vapor pressure between 0.001 Pa and 172 Pa and an aqueous solubility of at least about 24.99 grams organic acid per 100 grams water, both at room temperature and standard pressure. The device can generate droplets of the formulation having a median particle size (Dv50) of no greater than about 120 micrometers. In one embodiment of the invention, the formulation delivered as droplets in the air environment can be effective to neutralize greater than 50% of one or more airborne allergens present in the air environment.

In example embodiments, a system for neutralization of airborne allergens comprises a formulation comprising an organic acid having a vapor pressure between 0.001 Pa and 172 Pa at room temperature and standard pressure and an aqueous solubility of at least about 24.99 grams organic acid per 100 grams water at room temperature and standard pressure, a solvent selected from the group consisting of glycols, glycol ethers, glycol ether acetates and any combinations or mixtures thereof, and, optionally, one or more adjuvants selected from the group consisting of fragrances, waxes, dyes, colorants, stabilizers, thickeners, defoamers, hydrotropes, buffers, builders, enzymes, cloud point modifiers, preservatives, and any mixtures or combinations thereof. The system further comprises a device that generates droplets of the formulation having a median particle size (Dv50) of no greater than about 120 micrometers. The system provides a statistically significant reduction, over a control, in one or more airborne allergens as measured by an enzyme linked immunoassay.

In additional example embodiments, a system for neutralization of airborne allergens comprises a formulation comprising an organic acid having a vapor pressure between 0.001 Pa and 172 Pa at room temperature and standard pressure and an aqueous solubility of at least about 24.99 grams organic acid per 100 grams water at room temperature and standard pressure, The system further comprises a device that delivers the formulation as droplets to an air environment and in an amount effective to neutralize greater than 50% of one or more airborne allergens present in the air environment, e.g., within 15 minutes or about 1 to 20 minutes, from initial delivery of the formulation into the air environment.

In further example embodiments, a method of neutralizing airborne allergens within an air environment comprises generating droplets of a formulation via a device and delivering an effective amount of the droplets into an air environment. The formulation comprises an organic acid having a vapor pressure between 0.001 Pa and 172 Pa at room temperature and an aqueous solubility of at least about 24.99 grams organic acid per 100 grams water, and the effective amount of the droplets delivered into the air environment provides a statistically significant reduction, over a control, in one or more airborne allergens as measured by an enzyme linked immunoassay. The effective amount of the droplets delivered into the air environment can further neutralize greater than 50% of one or more airborne allergens present in the air environment, e.g., within 15 minutes from initial delivery of the formulation into the air environment.

The above and still further features and advantages of the present invention will become apparent upon consideration of the following detailed description of specific embodiments thereof.

### BRIEF DESCRIPTION OF THE DRAWINGS

FIG. 1 is a plot of comparative test data showing efficacy in neutralization of airborne allergens utilizing for a formulation including lactic acid vs. a formulation including citric acid.

FIG. 2 is a plot of comparative test data showing efficacy in neutralization of airborne allergens for a number of formulations including different organic acids.

FIG. 3 is a plot showing efficacy of anti-allergen formulations in neutralizing different types of airborne allergens.

FIG. 4 is a plot of statistically relevant data showing efficacy of an anti-allergen formulation tested a number of times against different airborne allergens.

### DETAILED DESCRIPTION

### I. Definitions

In the following detailed description, while aspects of the disclosure are disclosed, alternate embodiments of the present disclosure and their equivalents can be devised without parting from the spirit or scope of the present disclosure. It should be noted that any discussion herein regarding “one embodiment”, “an embodiment”, “an exemplary embodiment”, and the like indicate that the embodiment described can include a particular feature, structure, or characteristic, and that such particular feature, structure, or characteristic can not necessarily be included in every embodiment. In addition, references to the foregoing do not necessarily comprise a reference to the same embodiment. Finally, irrespective of whether it is explicitly described, one of ordinary skill in the art would readily appreciate that each of the particular features, structures, or characteristics of the given embodiments can be utilized in connection or combination with those of any other embodiment discussed herein.

Various operations can be described as multiple discrete actions or operations in turn, in a manner that is most helpful in understanding the claimed subject matter. However, the order of description should not be construed as to imply that these operations are necessarily order dependent. In particular, these operations cannot be performed in the order of presentation. Operations described can be performed in a different order than the described embodiment. Various additional operations can be performed and/or described operations can be omitted in additional embodiments.

For the purposes of the present disclosure, the phrase “A and/or B” means (A), (B), or (A and B). For the purposes of the present disclosure, the phrase “A, B, and/or C” means (A), (B), (C), (A and B), (A and C), (B and C), or (A, B and C).

The terms “comprising”, “including”, “containing”, “having” and “characterized by”, as used herein, are synonymous and are open-ended terms that do not exclude additional, unrecited elements or method steps. The term “consisting of”, as used herein, excludes any element or method step that is not specified in a claim. The term “consisting essentially of”, as used herein, limits the scope of a claim to specific recited elements and method steps and as well as unrecited elements and unrecited steps that do not materially affect the basic and novel characteristics of the claimed invention.

The terms “a”, “an” and “the” can refer to plural elements unless clearly indicated otherwise (e.g., “an excipient” can include, one, two or more excipients).

The term “effective amount”, as used herein, generally refers to amounts described within ranges as noted herein. Also, unless indicated otherwise, the term “percentage”, as used herein, refers to a weight percent based upon a particular material that is referenced. For example, an excipient, active ingredient or component that is present in an amount of 1% of a formulation or other composition indicates that the excipient, active ingredient or other component is present in the formulation or composition as 1% by total weight of the formulation or composition. Such term is also referred to herein as “wt %” or “% by weight”. Similarly, the term “ppm” refers to parts per million on a weight/weight basis, such that, e.g., 100 ppm refers to 0.01% by weight (or 0.01 wt %). The term “room temperature” refers to a temperature from about 20° C. to about 22° C. The term “standard pressure” refers to an atmospheric pressure in typical environments (e.g., households) that is about 1 atm (about 101.325 kPa). The term “range from”, “a range of”, or “between” is inclusive of any endpoints noted relative to a described range.

Further, the term “about” as used herein in relation to a described amount indicates that the amount can deviate or vary slightly beyond the described value by no more than 5% (e.g., “about 1% by weight” also includes a range of 0.95-1.05% by weight) while substantially maintaining the same efficacy of the formulation.

In addition, the term “formulation”, as used herein (e.g., an anti-allergen formulation), refers to an aqueous liquid composition including two or more ingredients or components (e.g., at least an active ingredient and water).

The term “free of” or similar phrases if used herein means that the formulation, composition or system comprises 0% of the stated component, that is, the component has not been intentionally added. However, it will be appreciated that such components can incidentally form thereafter, under some circumstances, or such component can be incidentally present, e.g., as an incidental contaminant. The phrase “substantially free of” or similar phrases as used herein means that the formulation, composition or system preferably comprises 0% of the stated component, although it will be appreciated that very small concentrations can possibly be present, e.g., through incidental formation, contamination, or even by intentional addition. Such components can be present, if at all, in amounts of less than 1%, less than 0.5%, less than 0.25%, less than 0.1%, less than 0.05%, less than 0.01%, less than 0.005%, less than 0.001%, or less than 0.0001%. In some embodiments, the formulations, compositions, or systems described herein can be free or substantially free from any specific components not mentioned within this specification.

Formulations and systems as described herein can provide inactivation or neutralization of airborne allergens by denaturing proteins of allergens in an airborne environment. Airborne allergens or airborne particles comprising allergens, as described herein, can comprise, e.g., pet dander (e.g., cat dander, dog dander or other companion animal dander), dust mites, various types of pollens (e.g., grass pollens, tree pollens, other plant pollens), mold spores, or any form of antigen that causes an allergic reaction or immune response within the body of a human or other mammal. The terms “neutralize”, “neutralizing”, and “neutralization” in relation to an airborne allergen refers to one or more proteins of the allergen being inactivated or denatured as a result of interactions with anti-allergen formulations as described herein, such that the allergen proteins are effectively prevented from interacting or binding with an immunoglobulin (Ig) receptor of the human or other mammal. As further described herein, formulations and systems as described herein are capable of neutralizing at least 20%, or at least 30%, or at least 40%, or at least 50%, or even greater than 50% of airborne allergens within an air environment after a select time period (e.g., within 5 minutes, within 10 minutes, within 15 minutes, within 20 minutes, etc.)) from initial delivery of the formulations into the air environment. In particular, a delivery device of the system can deliver an effective amount of an anti-allergen formulation to neutralize at least about 20%, or at least about 30%, or at least about 40%, or at least about 50%, or greater than about 50% of one or more airborne allergens present in an air environment (e.g., an enclosed air environment, such as a room, office, hallway, closet, etc.) after a select time period (e.g., within 5 minutes, within 10 minutes, within 15 minutes, within 20 minutes, etc.) of initial delivery of the formulations in the air environment. The air environment can be a closed air environment having a volume of at least about 1 m³.

The term “active ingredient”, as used herein, refers to an ingredient or component within the anti-allergen formulations described herein that are capable of neutralizing airborne allergens. The anti-allergen formulations as described herein can include one or more active ingredients and/or one or more ingredients or components that are not active ingredients (e.g., surfactants, fragrances, buffers and other adjuvants as described herein).

As further described herein, a measurement of neutralization of airborne allergens within a defined environment can be determined based upon sampling and detecting the presence of a number of active allergens (i.e., allergens which bind with Ig receptors) in air over a select time period using a conventional or other suitable enzyme linked immunoassay technique such as ELISA (Enzyme-Linked Immunosorbent Assay), MARIA (Multiplex Array for Indoor Allergens) and/or Mycometer (ELISA surrogate) test methods.

The formulations as described herein can also be prepared including ingredients or components recognized as safe according to the U.S. Environmental Protection Agency (EPA) Safer Choice criteria. In particular, ingredients or components utilized in the formulations described herein can be listed on the Safer Chemical Ingredients List as set forth by the EPA and as set forth at https://www.epa.gov/saferchoice/safer-ingredients #scil.

### II. Anti-Allergen Formulations and Systems

Anti-allergen formulations and corresponding systems and methods are described herein that are useful for neutralization of airborne allergens in a select environment. The environment can be an enclosed environment (e.g., any space or room in a residential or commercial environment). As described herein, to be effective for neutralization of airborne allergens, one or more active ingredients within the formulation should be capable of escaping or leaving aerosolized liquid droplets in order to effectively interact with and neutralize airborne allergens. In example embodiments, each active ingredient has a suitable vapor pressure at room temperature and standard pressure and the aerosolized liquid droplets are of a sufficient particle size to enable effective release of the active ingredient from the droplets into the environment for interaction with airborne allergens.

### Organic Acids in Anti-Allergen Formulations

The active ingredient(s) for the formulations include one or more organic acids effective in neutralizing allergens by denaturing proteins of the allergens as previously described herein. Organic acids useful for the formulations described herein have a vapor pressure ranging from about 0.001 Pa (Pascals) to about 172 Pa at room temperature (e.g., at a temperature ranging from about 20° C. to about 22° C.) and standard pressure (e.g., about 101.325 kPa). In other example embodiments, organic acids useful for formulations described herein have a vapor pressure ranging from about 0.4 Pa to about 172 Pa at room temperature and standard pressure. Organic acids having a vapor pressure within this range have sufficient volatility to be released or escape from aerosolized droplets in air. Organic acids particularly suitable for the formulations described herein, in addition to having the noted vapor pressure, should also be soluble in water. In particular, organic acids of interest have a suitable aqueous solubility that is at least about 24.99 g (grams) of organic acid in 100 g of water at room temperature and standard pressure. As further described herein, a suitable device is provided that delivers aerosolized droplets of formulation having a median particle size (Dv50) of no greater than about 120 micrometers (microns).

In example embodiments, the organic acid is a short chain organic acid, e.g., having no more than 12, no more than 10, no more than 8, or no more than 6 carbon atoms. The organic acid can be a mono-organic acid, a dicarboxylic, or a polycarboxylic acid. Some further non-limiting examples of organic acids that can be used as the active ingredient in the formulations described herein include alpha hydroxy acids, fatty acids, keto acids, dicarboxylic acids, unsaturated carboxylic acids, alkane sulfonic acids, and aromatic acids. An example organic acid that is effective for use as the active ingredient in the formulations described herein (e.g., meets the vapor pressure and aqueous solubility requirements at room temperature and standard pressure) is lactic acid. A non-limiting group of organic acids that can be useful as active ingredients in formulations described herein (having suitable vapor pressures and aqueous solubilities at room temperature and standard pressure) includes lactic acid, glycolic acid, butyric acid, pyruvic acid, maleic acid, and methane sulfonic acid. Formulations can include one or more of such organic acids and any combinations or mixtures thereof.

Each organic acid of interest (e.g., satisfying the vapor pressure and aqueous solubility requirements as previously noted herein) can be provided in an effective amount within a formulation to provide a sufficient neutralization of airborne allergens. For example, each organic acid can be included in the formulations described herein in an amount from about 0.05%, from about 0.1%, from about 0.2%, from about 0.5%, from about 1%, from about 1.5%, from about 2%, up to about 20%, up to about 15%, up to about 10%, up to about 8%, up to about 6%, up to about 5%, up to about 4%, or up to about 3% by weight of the formulation. In a further example, an effective amount of an organic acid (e.g., lactic acid) in the formulation can range from about 0.1% to about 20% by weight of the formulation, or from about 0.2% to about 20% by weight of the formulation. In a still further example, an effective amount of an organic acid in the formulation can range from about 0.1% to about 5% by weight of the formulation, or from about 1% to about 5% by weight of the formulation, or from about 1% to about 4.5% by weight of the formulation, or from about 1% to about 4.3% by weight of the formulation. In a specific example, lactic acid can be provided in a formulation in an effective amount of about 2% by weight of the formulation.

The anti-allergen formulations can include one or more (e.g., a plurality of) organic acids and water. The anti-allergen formulations can be substantially free of any other ingredient or component other than the one or more organic acids and water. Alternatively, in other embodiments, the anti-allergen formulations can include one or more further ingredients or components, such as one or more surfactants and/or one or more adjuvants as described herein. Furthermore, the anti-allergen formulations can be free of or substantially free of any active ingredient other than one or more organic acids having vapor pressures and solubilities within the ranges as described herein.

In some example embodiments, the pH of the anti-allergen formulations can be kept low. A low pH can enhance the organic acid efficacy (e.g., lactic acid efficacy) in neutralization of airborne allergens. Typically, pH is kept a least 1 unit below pKₐ of the organic acid to ensure majority (at least 90%) of acid stays in the active (conjugate acid) form. For example, lactic acid is most efficacious in the acid form compared to the conjugate base form (lactate salts), since the acid form of lactic acid is more volatile. At a pH of 2.2, at least 95% of present lactic acid is in the acidic form (lactic acid has a pKₐ of 3.79). While lactic acid has higher efficacy at low pH, a neutral pH formulation has better aesthetics and potentially lower toxicity. Therefore, a pH that provides a balance between efficacy of lactic acid with regard to neutralization of airborne allergens and aesthetics and toxicity levels is desirable.

By way of example, the pH can be at least about 1, at least about 1.5, at least about 2, up to about 6.5, up to about 6, up to about 5, up to about 4, up to about 3, up to about 2, up to about 2.5, or up to about 2.4. In example embodiments, the pH can be maintained between 2 and 4, or 2 and 3. The pH can be maintained, e.g., by the addition of a buffer such as citric acid or another suitable buffer including but not limited to: acids, bases, carbonates, bicarbonates, etc. However, it is important to note that the citric acid (when provided in formulations) may be present as a buffer, rather than for any providing any primary allergen neutralization benefit in airborne environments. The citric acid buffer can be included in an amount of at least about 0.05%, at least about 0.1%, up to about 6%, up to about 5%, up to about 4%, up to about 3%, up to about 2%, up to about 1.5%, or up to about 1% by weight of the anti-allergen formulation.

### Surfactants in Anti-Allergen Formulations

In example embodiments, the anti-allergen formulation can include one or more surfactants, such as one or more anionic surfactants and/or one or more nonionic surfactants. In some embodiments, one or more surfactants can be included in an amount of at least about 0.025%, at least about 0.05%, at least about 0.1%, at least about 0.2%, at least about 0.3%, at least about 0.4%, at least about 0.5%, at least about 1%, and up to about 10%, up to about 5%, up to about 3%, up to about 2%, or up to about 1%, by weight of the anti-allergen formulation. For example, one or more surfactants (e.g., one or more non-ionic surfactants) can be included in the anti-allergen formulations in an amount between about 0.5% and about 10% by weight of the formulation, or between about 1% and about 5% by weight of the formulation.

Those skilled in the art will appreciate that any among a wide variety of surfactants (e.g., anionic, cationic, non-ionic, zwitterionic, and/or amphoteric) can be included in the formulation, as desired. Where included, a surfactant can be present from about 0.05%, from about 0.1%, up to about 10%, up to about 5%, up to about 4%, up to about 3%, up to about 2%, or up to about 1% by weight of the formulation. Various surfactants and other optional adjuvants are disclosed in U.S. Pat. No. 3,929,678 to Laughlin and Heuring, U.S. Pat. No. 4,259,217 to Murphy, U.S. Pat. No. 5,776,872 to Giret et al.; U.S. Pat. No. 5,883,059 to Furman et al.; U.S. Pat. No. 5,883,062 to Addison et al.; U.S. Pat. No. 5,906,973 to Ouzounis et al.; U.S. Pat. No. 4,565,647 to Llenado, and U.S. Publication No. 2013/0028990. The above patents and applications are each herein incorporated by reference in their entirety.

Examples of nonionic surfactants include, but are not limited to, alcohol ethoxylates, alcohol propoxylates, other alcohol alkoxylates including fatty (e.g., C₆, C₈, C₁₀, or C₁₂, or higher) alcohols or other constituents that have been alkoxylated to include both ethoxy and propoxy groups (EO-PO surfactants), alkyl phosphine oxides, alkyl glucosides and alkyl pentosides, alkyl glycerol esters, alkyl ethoxylates, and alkyl and alkyl phenol ethoxylates of all types, poly alkoxylated (e.g. ethoxylated or propoxylated) C₆-C₁₂ linear or branched alkyl phenols, C₆-C₂₂ linear or branched aliphatic primary or secondary alcohols, and C₂-C₈ linear or branched aliphatic glycols. Block or random copolymers of C₂-C₆ linear or branched alkylene oxides can also be suitable nonionic surfactants. Capped nonionic surfactants in which the terminal hydroxyl group is replaced by halide; C₁-C₈ linear, branched or cyclic aliphatic ether; C₁-C₈ linear, branched or cyclic aliphatic ester; phenyl, benzyl or C₁-C₄ alkyl aryl ether; or phenyl, benzyl or C₁-C₄ alkyl aryl ester can also be used. Sorbitan esters and ethoxylated sorbitan esters can also be useful nonionic surfactants. Other suitable nonionic surfactants can include mono or polyalkoxylated amides of the formula R¹CONR²R³ and amines of the formula R¹NR²R³ wherein R¹ is a C₅-C₃₁ linear or branched alkyl group and R² and R³ are C₁-C₄ alkyl, C₁-C₄ hydroxyalkyl, or alkoxylated with 1-3 moles of linear or branched alkylene oxides. Biosoft 91-6 (Stepan Co.) is an example of an alkyl ethoxylate (or alcohol ethoxylate) having a methylene chain length of C₉ to C₁₁ with an average of 6 moles of ethoxylation. An example of an alcohol ethoxylate is ECOSURF EH-9, which is more specifically an ethylene oxide-propylene oxide copolymer mono(2-ethylhexyl) ether, available from Sigma-Aldrich.

Alkylpolysaccharide nonionic surfactants are disclosed in U.S. Pat. No. 4,565,647 to Llenado, having a linear or branched alkyl, alkylphenyl, hydroxyalkyl, or hydroxyalkylphenyl group containing from 6 to 30 carbon atoms and a polysaccharide, e.g., a polyglycoside, hydrophilic group containing from 1.3 to 10 saccharide units. Suitable saccharides can include, but are not limited to, glucosides, galactosides, lactosides, and fructosides. Alkylpolyglycosides can have the formula: R²O(CnH₂ₙO)ₜ(glycosyl)ₓ wherein R² is selected from the group consisting of alkyl, alkylphenyl, hydroxyalkyl, hydroxyalkylphenyl, and mixtures thereof in which the alkyl groups contain from 10 to 18 carbon atoms; n is 2 or 3; t is from 0 to 10, and x is from 1.3 to 10.

Fatty acid saccharide esters and alkoxylated fatty acid saccharide esters can also be suitable for use in the present invention. Examples include, but are not limited to, sucrose esters, such as sucrose cocoate, and sorbitan esters, such as polyoxyethylene (20) sorbitan monooleate and polyoxyethylene (20) sorbitan monolaurate.

Phosphate ester surfactants can also be suitable. These include mono, di, and tri esters of phosphoric acid with C₄-C₁₈ alkyl, aryl, alkylaryl, alkyl ether, aryl ether and alkylaryl ether alcohols (e.g. disodium octyl phosphate).

Zwitterionic surfactants can be suitable. As zwitterionic surfactants include both a positive and negative functional group, they can also be classified as nonionic surfactants. Many such zwitterionic surfactants contain nitrogen. Examples of such include amine oxides, sarcosinates, taurates and betaines. Examples include C₈-C₁₈ alkyldimethyl amine oxides (e.g., octyldimethylamine oxide, lauryldimethylamine oxide (also known as lauramine oxide), and cetyldimethylamine oxide), C₄-C₁₆ dialkylmethylamine oxides (e.g. didecylmethylamine oxide), C₈-C₁₈ alkyl morpholine oxide (e.g. laurylmorpholine oxide), tetra-alkyl diamine dioxides (e.g. tetramethyl hexanane diamine dioxide, lauryl trimethyl propane diamine dioxide), C₈-C₁₈ alkyl betaines (e.g. decylbetaine and cetylbetaine), C₈-C₁₈ acyl sarcosinates (e.g. sodium lauroylsarcosinate), C₈-C₁₈ acyl C₁-C₆ alkyl taurates (e.g. sodium cocoylmethyltaurate), C₈-C₁₈ alkyliminodipropionates (e.g. sodium lauryliminodipropionate), and combinations thereof. Lauryl dimethyl amine oxide (Ammonyx LO) myristyl dimethyl amine oxide (Ammonyx MO), decylamine oxide (Ammonyx DO) are examples of suitable zwitterionic surfactants, available from Stepan Co.

Non-limiting examples of anionic surfactants include alkyl sulfates (e.g., C₈-C₁₈ linear or branched alkyl sulfates such as sodium lauryl sulfate (SLS), and sodium tetradecylsulfate), linear alkylbenzene sulphonic acids or sulfonates (HLAS), alkyl sulfonates (e.g., C₆-C₁₈ linear or branched alkyl sulfonates such as sodium octane sulfonate and secondary alkane sulfonates, alkyl ethoxysulfates, fatty acids and fatty acid salts (e.g., C₆-C₁₆ fatty acid soaps such as sodium laurate), and alkyl amino acid derivatives. Other examples can include sulfate derivatives of alkyl ethoxylate propoxylates, alkyl ethoxylate sulfates, alpha olefin sulfonates, C₆-C₁₆ acyl isethionates (e.g. sodium cocoyl isethionate), C₆-C₁₈ alkyl, aryl, or alkylaryl ether sulfates, C₆-C₁₈ alkyl, aryl, or alkylaryl ether methyl sulfonates, C₆-C₁₈ alkyl, aryl, or alkylaryl ether carboxylates, sulfonated alkyldiphenyloxides (e.g. sodium dodecyldiphenyloxide disulfonate), and the like.

More specific examples of nonionic and/or zwitterionic surfactants include lauryl dimethyl amine oxide (Ammonyx LO), also known as lauramine oxide, myristyl dimethyl amine oxide (Ammonyx MO), decylamine oxide (Ammonyx DO), other amine oxides, any betaines, linear alcohol ethoxylates, secondary alcohol ethoxylates, alcohol propoxylates, alkyl polyglucosides, and combinations thereof.

In embodiments in which one or more certain organic acids (e.g., lactic acid) are provided in the formulations, one or more of the following surfactants are particularly useful when provided in formulations for denaturing airborne allergens: a non-ionic secondary alcohol ethoxylate surfactant (e.g., TERGITOL™ 15-S-9, commercially available from Dow Inc.), a non-ionic alkyl polyglucoside surfactant (e.g., GLUCOPON® 215 UP, commercially available from BASF Corporation), and an amphoteric lauramine oxide surfactant (AMMONYX® LO, commercially available from Stepan Company).

### Solvents for Anti-Allergen Formulations

The anti-allergen formulations can be free, or substantially free, of solvents. Alternatively, in example embodiments, the anti-allergen formulations can include one or more solvents, such as alcohol solvents (e.g., mono-alcohols or glycols) and/or glycol ether solvents. Example alcohol solvents include, without limitation, ethylene glycol, propylene glycol, dipropylene glycol, triethylene glycol, hexanediol, and hexylene glycol, and lower alcohol solvents (e.g., C₁-C₄ mono-alcohols). The amount of such alcohol solvents may be from about 1% to about 60%, or limited, e.g., to less than about 50%, less than about 40%, less than about 30%, less than about 20%, less than about 10%, less than about 5%, less than about 3%, less than about 2%, less than about 1%, less than about 0.5%, or less than about 0.3% by weight. Example glycol ether solvents include, without limitation, ethylene glycol monopropyl ether, ethylene glycol monobutyl ether, ethylene glycol monohexyl ether, ethylene glycol phenyl ether, propylene glycol n-propyl ether, propylene glycol monobutyl ether, propylene glycol t-butyl ether, diethylene glycol monoethyl or monopropyl or monobutyl ether, di- or tri-polypropylene glycol methyl or ethyl or propyl or butyl ether, acetate and/or propionate esters of glycol ethers. A glycol ether or other solvent may be included in an amount from at least about 0.1%, or about 0.25%, and up to about 5%, up to about 4%, up to about 3%, up to about 2%, and/or up to about 1% by weight of the anti-allergen formulations.

### Additional Adjuvants/Adjuncts for Anti-Allergen Formulations

The formulations can optionally include and/or be used in combination with one or more additional adjuvants or adjuncts. The adjuncts include, but are not limited to, fragrances or perfumes, waxes, dyes and/or colorants, solubilizing materials, stabilizers, thickeners, defoamers, hydrotropes, buffers (as previously noted herein), builders, lotions and/or mineral oils, enzymes, cloud point modifiers, preservatives, and/or chaotropic agents. In example embodiments, buffering and pH adjusting agents, when used, include, but are not limited to, organic acids (e.g., citric acid), mineral acids, alkali metal and alkaline earth salts of citrate, silicate, metasilicate, polysilicate, borate, carbonate, carbamate, phosphate, polyphosphate, pyrophosphates, triphosphates, tetraphosphates, ammonia, hydroxide, monoethanolamine, monopropanolamine, diethanolamine, dipropanolamine, triethanolamine, and/or 2-amino-2-methylpropanol.

One or more buffering agents can comprise low molecular weight, organic or inorganic materials used for maintaining the desired pH. For buffers that can be used, see McCutcheon's EMULSIFIERS AND DETERGENTS, North American Edition, 1997, McCutcheon Division, MC Publishing Company which is incorporated herein by reference. In other alternative embodiments, solubilizing materials, when used, can include, but are not limited to, hydrotropes (e.g., water soluble salts of low molecular weight organic acids such as the sodium and/or potassium salts of xylene sulfonic acid). As previously noted herein, a citric acid buffer can be provided in the amounts as previously indicated to maintain a low-level pH for particular formulations.

In still another and/or alternative embodiments, thickeners, when used, include, but are not limited to, polyacrylic acid, xanthan gum, calcium carbonate, aluminum oxide, alginates, guar gum, methyl, ethyl, clays, and/or propylhydroxycelluloses. In yet another and/or alternative embodiment, defoamers, when used, include, but are not limited to, silicones, amino silicones, silicone blends, and/or silicone/hydrocarbon blends. In still a further and/or alternative embodiment, preservatives, when used, include, but are not limited to, mildewstats or bacteriostats, methyl, ethyl and propyl parabens, short chain organic acids (e.g., acetic, lactic and/or glycolic acids), bisguanidine compounds (e.g., Dantagard and/or Glydant) and/or short chain alcohols (e.g., ethanol and/or IPA). In one aspect of this embodiment, the mildewstats or bacteriostats include, but are not limited to, mildewstats (including non-isothiazolone compounds) include Kathon GC, a 5-chloro-2-methyl-4-isothiazolin-3-one, Kathon ICP, a 2-methyl-4-isothiazolin-3-one, and a blend thereof, and Kathon 886, a 5-chloro-2-methyl-4-isothiazolin-3-one, all available from Rohm and Haas Company; Bronopol, a 2-bromo-2-nitropropane-1,3-diol, from Boots Company Ltd.; Proxel CRL, a propyl-p-hydroxybenzoate, from ICI PLC; Nipasol M, an o-phenyl-phenol, Na+ salt, from Nipa Laboratories Ltd.; Dowicide A, a 1,2-Benzoisothiazolin-3-one, from Dow Chemical Co.; and Irgasan DP 200, a 2,4,4′-trichloro-2-hydroxydiphenylether, from Ciba-Geigy A.G.

The amounts of each of such adjuvants (e.g., preservatives, etc.) can be provided in the formulations from at least about 0.025%, at least about 0.05%, at least about 0.1%, at least about 0.2%, at least about 0.25%, at least about 0.3%, at least about 0.4%, or at least about 0.5%, and up to about 10%, up to about 5%, up to about 3%, up to about 2%, up to about 1%, up to about 0.5%, up to about 0.4%, up to about 0.3%, or up to about 0.2% by weight of the formulations.

Fragrances can also be included in an amount of at least about 0.025%, at least about 0.05%, at least about 0.10%, up to about 1%, or up to about 0.5% by weight of the formulation. For example, fragrances can be added in an amount from about 0.25% to about 20% by weight of the formulation, or from about 0.1% to about 0.5% by weight of the formulation, or from about 0.25% to about 0.4% by weight of the formulation. Non-limiting examples of suitable fragrances include essential oils (e.g., from botanical or other naturally derived sources), synthetic fragrance oils and/or any other suitable type of fragrance composition that is stable at the concentration level of the organic acid and/or other components in the formulations, where the fragrance compositions are preferably rendered substantially miscible in the aqueous based formulations. In example embodiments, a fragrance can be provided in an amount from about 0.25% to about 0.35% by weight of the formulation.

In example embodiments (e.g., embodiments in which little or no solvent is provided), the formulations can include a majority of water, such as water that is present in an amount of at least about 50%, at least about 55%, at least about 60%, at least about 65%, at least about 70%, at least about 75%, at least about 80%, at least about 85%, or at least about 90% or greater by weight of the formulation. Such water can be present in free, unbound form. In embodiments in which solvent is provided, e.g., in an amount as great as about 60% by weight of the formulation, water can be present in an amount less than about 50% by weight of the formulation (e.g., water added in an amount as needed to make up the final formulation).

The formulations can have relatively low viscosities, e.g., similar to that of water, in order to facilitate dispensing and formation of small airborne droplets. For example, the formulation can have a viscosity of up to 1000 cps, up to 500 cps, or up to 100 cps, such as from 1 cps to 100 cps.

Some example embodiments of anti-allergen formulations that are effective in denaturing airborne allergens and which comprise lactic acid as the organic acid active ingredient are provided in Tables 1-3:

TABLE 1
Example anti-allergen formulation including
greater than 75% water by weight

| Ingredient/Component | Wt % in Formulation |
|---|---|
| Organic acid (lactic acid) | 1-5 |
| Preservative (sodium benzoate) | 0.05-0.2 |
| Non-ionic alkyl polyglucoside | 0.3-1.8 |
| surfactant (Glucopon 215 UP) | |
| Non-ionic secondary alcohol | 0.1-1 |
| ethoxylate surfactant (Tergitol 15-S-9) | |
| Amphoteric lauramine oxide | 0.1-0.6 |
| surfactant (AMMONYX ® LO) | |
| Fragrance | 0.25-0.4 |
| Deionized water | QS |

TABLE 2
Example anti-allergen formulation including
greater than 75% water by weight

| Ingredient/Component | Wt % in Formulation |
|---|---|
| Organic acid (lactic acid) | 1-5 |
| Preservative (sodium benzoate) | 0.05-0.2 |
| Non-ionic alkyl polyglucoside | 0.3-1.8 |
| surfactant (Glucopon 215 UP) | |
| Non-ionic secondary alcohol | 0.1-1 |
| ethoxylate surfactant (Tergitol 15-S-9) | |
| Fragrance | 0.25-0.4 |
| Deionized water | QS |

TABLE 3
Example anti-allergen formulation (including formulations
with less than 50% water by weight)

| Ingredient/Component | Wt % in Formulation |
|---|---|
| Organic acid (lactic acid) | 2-10 |
| Preservative (sodium benzoate) | 0.05-0.2 |
| Solvent (dipropylene glycol methyl | 0.1-25 |
| ether) | |
| Solvent (dipropylene glycol) | 0.1-35 |
| Amphoteric lauramine oxide | 0.1-0.6 |
| surfactant (AMMONYX ® LO) | |
| Fragrance | 0.25-20 |
| Deionized water | QS |

### Airborne Delivery Systems for Anti-Allergen Formulations

The anti-allergen formulations as described herein can be delivered as an aerosol or mist in the air environment to be treated. To effectively generate and deliver the formulations, a number of technical parameters are taken into consideration such as temperature, relative humidity, flow rate, particle size, density, vapor pressure, and other potential parameters. For example, smaller particle size is often correlated with relatively lower flow rates in lower cost delivery methods. Due to this, there is a need to balance particle size and flow rate for desired efficacy. Advantageously, the present systems, methods and formulations do not require expensive delivery equipment in order to achieve an aerosolized mist of the formulations that is effective in neutralization of airborne allergens.

For example, existing systems often require use of an expensive thermal fogger device in order to be effective. While such a device can of course be used to deliver the present formulations, such is not necessary, and treating a given air space with the anti-allergen formulations can be achieved through simple inexpensive delivery tools, such as through a trigger sprayer, trigger mist device, etc.

A variety of devices can be used to deliver the formulations. For example, some embodiments can use a pump sprayer, a HVLP sprayer, an aerosolizer, a mister, a nebulizer, a vaporizer, a ULV fogger, a cold fogger, a thermal fogger, an ultrasonic dispensing device, an electrostatic sprayer, plug-in dispensing devices, or combinations thereof. Different devices can generate and provide different particle size ranges of the delivered formulations into an airborne environment. Some devices, such as aerosols and pump sprayers, are low cost and easy to use. Other devices, such as foggers (particularly a thermal fogger), can have better performance (smaller particle size), but they can also be bulky, expensive, and may require training or professional use or personal protective equipment (PPE).

The total number of moles of organic acid within the formulation to be delivered to the air during an application can vary based upon a particular scenario. In example embodiments, the number of moles for formulation delivered into an air environment can be at least about 0.05 mmol/m³, at least about 0.060 mmol/m³, at least about 0.075 mmol/m³, at least about 0.1 mmol/m³, at least about 0.2 mmol/m³, at least about 0.3 mmol/m³, at least about 0.4 mmol/m³, or at least about 0.5 mmol/m³, or at least about 0.6 mmol/m³, and up to about 5 mmol/m³, up to about 4 mmol/m³, up to about 3 mmol/m³, up to about 2.5 mmol/m³, up to about 2 mmol/m³, or up to about 1 mmol/m³.

For ensured effectiveness in neutralization of airborne allergens, the formulation must be delivered in droplets having a median particle size (Dv50) of no greater than about 120 μm (micrometers, or microns). Any conventional or other suitable method can be utilized for determining particle size distribution, and thus the median particle size (Dv50) for droplets of formulation delivered by a suitable delivery device. In an example embodiment, the particle size distribution of droplets of formulation formed by a device can be measured with a laser diffraction system, such as a laser diffraction system commercially available under the tradename SPRAYTEC (Malvern Panalytical). For a delivery devices that generates a horizontal spray, particle size measurements can be obtained by placing the delivery device about 6 inches (about 15.24 cm) from the laser of the laser diffraction system, with the spray being emitted perpendicular to the laser and at a suitable height so that the laser intersects the center of the spray. For a delivery device which generates a vertical spray, the deliver device can be placed directly below the laser of the laser diffraction system and at about 6 inches (about 15.24 cm) from the laser. Delivery devices that produce particularly small particle sizes (e.g., around 10 μm or less), the device can be positioned closer to the laser of the laser diffraction system to accurately measure the particle size before the spray evaporates.

In particular, the formulation can be delivered in droplets having a median particle size (Dv50) of no greater than about 100 μm, or no greater than about 70 μm, or ranging between about 5 μm and about 70 μm. In certain embodiments, the median particle size (Dv50) of droplets of formulation can even be less than about 5 μm. The delivery devices described herein are capable of providing aerosolized droplets of the anti-allergen formulations predominately within such particle size ranges.

### III. Methods for Application/Delivery of Anti-Allergen Formulations

The amount of anti-allergen formulation to be sprayed within an enclosed environment will be based upon particular formulation, particular delivery device including flow rate of droplets from the delivery device at the select size (median particle size by volume (Dv50) no greater than 120 μm), and size (volume) of enclosed environment.

For example, consider a scenario in which a trigger pump spray device is used to deliver the anti-allergen formulation into the room in droplets at the select droplet size. For a room within a residential environment (e.g., bedroom, living room, etc.) having a size of about 8 ft (2.4 m)×10 ft (3 m)×9 ft height (2.7 m height), or a volume of about 720 ft3 (about 19 m³), the hand-held trigger spray device can be activated by manually engaging (i.e., pulling back or actuating) the trigger a plurality of times, e.g., from at least two trigger engagement sprays to no more than ten trigger engagement sprays. This process can be repeated as deemed appropriate for a particular scenario.

Alternatively, when employing an automated delivery device, such as a mister or fogger, the device can be selectively controlled to deliver the anti-allergen formulation at the select droplet size and selected flow rate over select time periods. For example, an automated delivery device can automatically deliver a spray of anti-allergen formulation at the suitable droplet size and selected amount for a given room size at select time intervals (e.g., one or more sprays over a select number of minutes or hours) throughout a given time period (e.g., daily, hourly, etc.).

An effective amount of the droplets of formulation delivered at a suitable particle size distribution into the air environment provides a statistically significant reduction, over a control, in one or more airborne allergens, and this can be determined via measurement using an enzyme linked immunoassay (e.g., ELISA assay or MARIA assay). As used herein, the term “statistically significant reduction” with regard to efficacy of formulation in comparison to control (i.e., no formulation), refers to maintaining repeatability of the amount of reduction of airborne allergen treated with formulation under the same or similar conditions in comparison to any reduction of airborne allergen that may occur in the absence of formulation. In particular, when performing a number of treatments of a particular airborne allergen vs. no treatment under the same or substantially similar conditions (e.g., airborne environment volume, temperature, amount of airborne allergen initially present, amount of formulation provided in the airborne environment volume, and time period for determination of airborne allergen amount after an initial determination of amount of airborne allergen in which no formulation is present), there is a statistically significant difference in the mean of the data associated with the amount of allergen remaining after a set time period for formulation treatment vs. the amount of allergen remaining after the set time period and without any treatment of formulation. To determine if there is a statistically significant difference in the means, a suitable statistical test for comparing means of distributions, such as a 2-sample t-test, should be employed, which accounts for the standard deviations of the measurements.

### IV. EXAMPLES

### Example 1—Effective Organic Acids for Anti-Allergen Formulations

As previously described herein, formulations that are particularly effective in neutralization of airborne allergens are those having a suitable vapor pressure at room temperature (about 20° C. to about 22° C.) and standard pressure (about 1 atm, or about 101.325 kPa) that ranges from about 0.001 Pa (Pascals) to about 172 Pa, or from about 0.4 Pa to about 172 Pa. The organic acid should further be soluble in water, preferably having an aqueous solubility that is at least about 24.99 g (grams) of organic acid in 100 g of water at room temperature and standard pressure. Some non-limiting examples of organic acids, including examples of particularly suitable organic acids meeting this criteria and examples of organic acids that are not well suited for the anti-allergen formulations described herein, are set forth in Table 4:

TABLE 4
Listing of organic acids

| | | | Solubility in | |
|---|---|---|---|---|
| | | | water (g of | Suitable for anti- |
| | | Vapor | compound in | allergen formulation |
| | | Pressure | 100 g of water | (meets vapor pressure |
| Category | Organic Acid | (Pa) | at ~20° C.) | and solubility criteria) |
| alpha hydroxy | lactic acid | 10.00 | Miscible | Y |
| acid | | | | |
| | glycolic acid | 0.41 | 30 | Y |
| | citric acid | 0.000002 | 59.2 | N |
| fatty acid | acetic acid | 2080 | 60.29 | N |
| | propionic acid | 390 | 100 | N |
| | butyric acid | 100 | Very soluble | Y |
| | valeric acid | 100 | 3.75 | N |
| | hexanoic acid | 5.80 | 1.03 | N |
| | heptanoic acid | 1.35 | 0.2 | N |
| | octanoic acid | 0.49 | 0.068 | N |
| Alkane sulfonic | methane | 0.048 | 100 | Y |
| acid | sulfonic acid | | | |
| keto acid | levulinic acid | 0.374 | 79.13 | Y |
| | pyruvic acid | 172 | 100 | Y |
| dicarboxylic | adipic acid | 9.70 | 23 | Y |
| acid | | | | |
| | malonic acid | 0.00049 | 76.3 | N |
| unsaturated | fumaric acid | 0.02 | 0.7 | N |
| carboxylic acid | | | | |
| | maleic acid | 0.001 | 47.88 | Y |
| | malic acid | 0.000004 | 64.7 | N |
| | sorbic acid | 0.018 | 0.156 | N |
| aromatic acid | benzoic acid | 0.11 | 0.35 | N |
| amino acid | glycine | 0.000017 | 24.99 | N |

Thus, a number of organic acids listed in Table 4 have vapor pressure and solubility characteristics that would render formulations including such organic acids as being effective in neutralization of airborne allergens, particularly when the formulations are aerosolized by a delivery device to droplets having a median particle size (Dv50) of no greater than about 120 μm.

In certain embodiments, effective anti-allergen formulations including organic acids meeting the vapor pressure and solubility requirements as noted herein can also meet a European Chemicals Agency (ECHA) long term inhalation threshold limit for the general population of at least 20 mg/m³. Further, effective anti-allergen formulations can be formed entirely from components that are fall within the Safer Choice criteria as set forth by the EPA. For example, formulations including lactic acid at a concentration between 1% and 5% by weight of the formulation meets EPA Safer Choice criteria as well as the ECHA long term inhalation threshold limit.

### Example 2—Tests Showing Efficacy of Anti-Allergen Formulations

The efficacy of certain formulations (containing organic acids as described herein and listed in Tables 1-3) in neutralization of airborne allergens can be determined based upon test methods now described. In particular, neutralization efficacy is determined based upon success of formulations in denaturing proteins in airborne allergens, where air in an enclosed environment is sampled before and after application of the formulations (via delivery device) and the extracted air is analyzed utilizing ELISA (Enzyme-Linked Immunosorbent Assay), MARIA (Multiplex Array for Indoor Allergens) and/or ELISA surrogate test methods. There may be variation in the type and size of an enclosed environment, the timing for the collecting the sample and the exact method of collection, but as long as the sample size is sufficient to an ELISA or MARIA assay the applicable detection limits for the allergen being tested will apply. In particular, a suitable ELISA surrogate test method that can be used is an air allergen test method and related test equipment commercially available under the trademark MYCOMETER (Mycometer Inc.) (also referred to herein as “Mycometer assay”). The detection limits utilizing ELISA methods can range from about 0.2 ng/ml to about 4 ng/nl depending upon particular allergen assayed, while MARIA methods has even greater sensitivity with detection limits ranging from about 0.01 ng/ml to about 0.98 mg/nl depending upon allergen assayed (further information regarding such methods is available at https://inbio.com). There may be variation in the type and size of an enclosed environment, the timing for the collecting the sample and the exact method of collection, but as long as the sample size is sufficient to an ELISA or MARIA assay the applicable detection limits for the allergen being tested will apply.

The Mycometer assay utilizes Enzyme Targeted Fluorogenic Detection (ETFD) to identify naturally occurring enzyme activities that are present only in a taxonomic group of interest. Thus, the Mycometer assay for testing efficacy of anti-allergen formulations tests for the presence and quantity of active allergen proteins (e.g., from mold spores, dust mites, pet dander, etc.), thus identifying presence and quantity of airborne allergens in a particular environment in which air is sampled. The enzyme activity is determined using highly sensitive fluorescence detection, where the fluorometric assay detects beta-N acetyl hexosaminidase (NAHA) activity (which is a marker for airborne allergens). The Mycometer assay utilizes a synthetic enzyme substrate that reacts with NAHA present in the allergen particle to release a fluorophore allowing detection with a handheld fluorometer. The amount of fluorescence correlates with amount of airborne allergen present in the tested environment.

Air can be sampled within an environment both before and after application of the formulation, via the delivery device, in a suitable amount within the environment. Reduction in detected allergen proteins after application of formulation provides an indication of efficacy in neutralization of airborne allergens via the formulation being tested.

In the test methods, a solution containing a surrogate protein in deionized (DI) water (0.5% by weight protein solution) is nebulized in a 1 m³ chamber for 15 minutes at 15 psi (103.4 kPa). After nebulization, the air is sampled every 5 minutes for a total of 20 minutes. At each collection time (0 min, 5 min, 10 min, 15 min, 20 min), the air is sampled for 1 minute at a flow rate of 10 LPM (liters per minute) using an air filter for the Mycometer assay. The airborne protein is extracted from the filter and analyzed in the Mycometer assay to provide a set of baseline measured values (i.e., representing airborne allergen content in the test chamber prior to treatment with formulation).

After nebulization of the protein solution, an anti-allergen formulation containing an organic acid is sprayed in the quantity of 0.67 mmol/m³. Each formulation contained an organic acid in an amount between 1% and 5% by weight of the formulation. The delivery device (nebulizer) delivers each tested formulation into the test chamber in droplet form having a median size (Dv50) for droplets between 5 μm and 70 μm. The air collection and analysis is performed in the same manner as previously described prior to application of the formulation in the 1 m³ chamber.

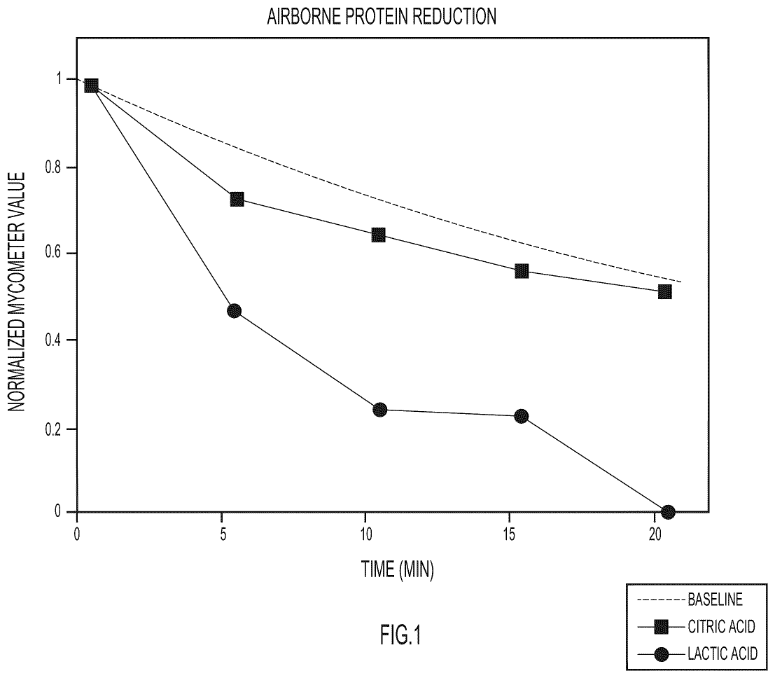

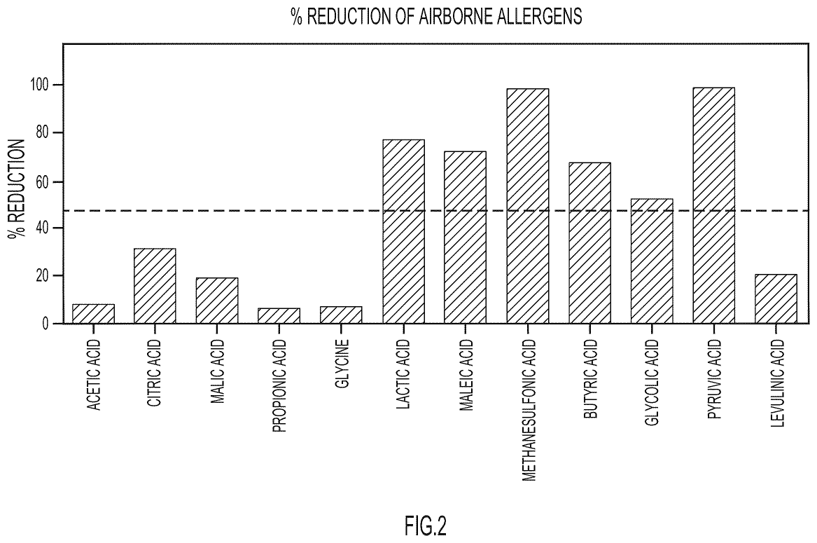

The test results using the Mycometer assay for formulations tested are provided in FIGS. 1 and 2.

Referring to FIG. 1, a comparison of the test results for a lactic acid formulation and a citric acid formulation is plotted along with the measured values representing the baseline. As indicated by the data plotted in FIG. 1, while both formulations provide some denaturing of allergen protein/neutralization of airborne allergen (i.e., both plots fall below the baseline), the lactic acid formulation has a much greater efficacy, providing a significant reduction in active protein (i.e., due to the denaturing of the protein in air) within 15 minutes of initial application/delivery of the formulation into the test chamber, which represents a neutralization of airborne allergen of greater than 50% (greater than about 70%) within 15 minutes of initial application. In contrast, the citric acid formulation was only slightly effective, providing a neutralization of airborne allergen that was significantly less than 50% (even less than 40%) within and even after 15 minutes of application.

Mycometer assay test data for further formulations including different organic acids is provided in FIG. 2. For each organic acid formulation, the % reduction or % neutralization in airborne allergen (based upon change from baseline) over a set time period (e.g., up to 20 minutes) from initial application is plotted for each formulation. A horizontal dotted line is provided at 50% reduction, clearly indicating which organic acid formulations have the greatest efficacy, namely, a reduction or neutralization of airborne allergens of greater than 50% within 15 minutes of initial application of the formulation into the test chamber. In particular, of the formulations tested, the formulations including one of lactic acid, maleic acid, methane sulfonic acid, butyric acid, pyruvic acid, and glycolic acid all provide an efficacy of greater than 50% reduction or neutralization of airborne allergens within 15 minutes of application of the formulation in the air environment.

### Example 3—Efficacy of Anti-Allergen Formulations for Different Allergens

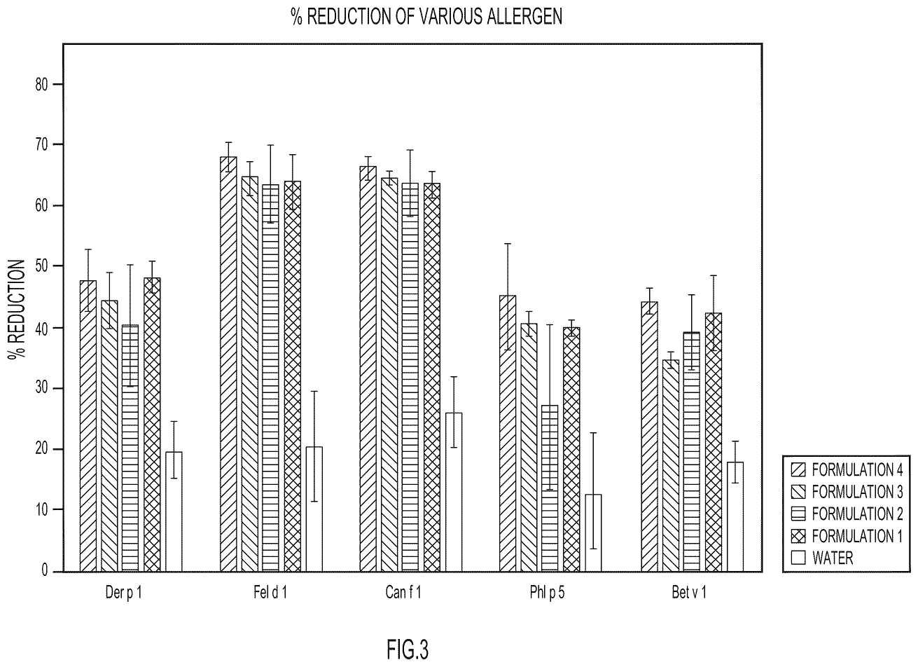

As demonstrated in this example, the efficacy of anti-allergen formulations as described herein are effective in neutralizing various types of airborne allergens. Formulations were tested against different airborne allergens, where the formulations included 2% by weight lactic acid. The formulations further included a preservative (sodium benzoate), a fragrance, and one or more surfactants as listed in Table 1 previously described herein (Glucopon, Tergitol and/or AMMONYX surfactants). Formulations were varied slightly based upon amount and type of components provided in each formulation, as shown in the data plotted in FIG. 3 (where the amount of each component was within the range set forth in Table 1). A water spray, provided as a control, was also tested against each airborne allergen. The airborne allergens tested included house dust mite allergen (Der p 1), cat allergen (Fel d 1), canine allergen (Can f 1), grass pollen allergen (Phl p 5), and birch pollen allergen (Bet v 1).

The data plotted in FIG. 3 shows the neutralizing efficacy as a % reduction after the application of each formulation vs. control (no treatment) when sprayed in an airborne environment. In particular, 25 grams of allergen-containing dust was initially dispersed into a 28 m³ chamber. For 40 minutes, the air was sampled continuously to determine the exact amount of measurable active allergen. In subsequent tests, the same amount of allergen is dispersed, and 65 grams of formulation is released 2 minutes thereafter. Air was sampled for the same 40 minutes, and the total amount of active allergen was compared to the control. MARIA testing was performed to determine amount of active allergen collected via the air sampling. As shown in the data of FIG. 3, the formulations were similar in their overall efficacy in reducing the same type of airborne allergen.

### Example 4-Efficacy of Anti-Allergen Formulation Providing Statistically Relevant Results for Different Allergens

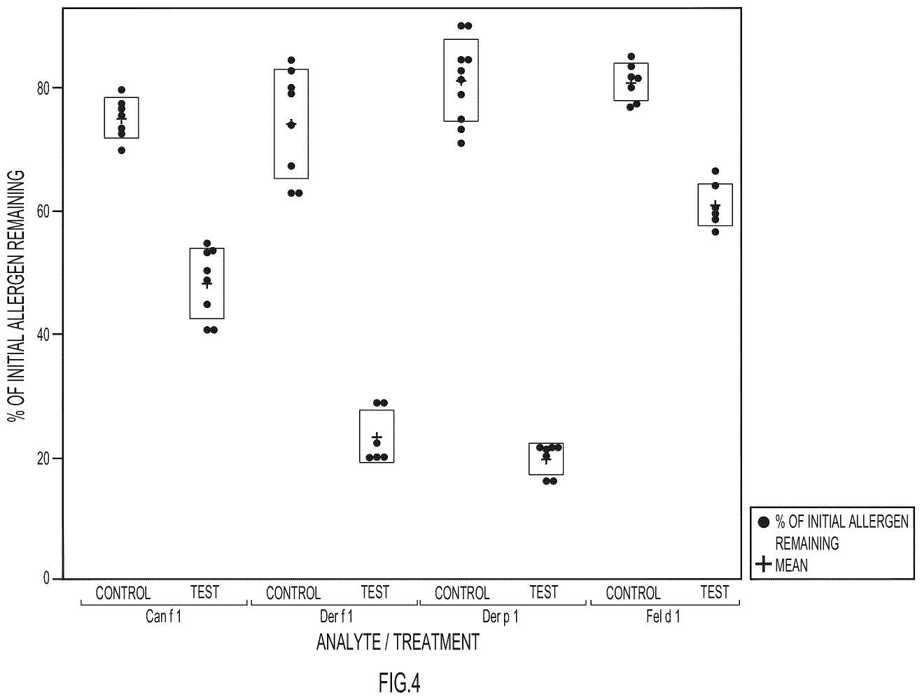

The same types of airborne allergens were used for testing a single formulation with a composition including 2% by weight lactic acid and further including other components provided in weight percentages within the ranges as set forth in Table 2. Tests using the same formulation were conducted, in which a 1 m³ chamber was used, a set amount of airborne allergen was provided within the chamber, and MARIA testing was performed to determine the initial amount of each airborne allergen present in the chamber and amount of each airborne allergen present in the chamber 10 minutes after the addition of the formulation to the test chamber. The test results are shown in the data plotted in FIG. 4.

For each baseline test (identified as “Control” in FIG. 4), no formulation was added to the chamber, and a measurement of % of initial allergen remaining was determined via MARIA testing 10 minutes after measuring the initial amount of the airborne allergen that was provided in the chamber. For each efficacy test, 1 g/m³ of formulation was sprayed into the chamber immediately after the measurement of the initial amount of airborne allergen added into the chamber had completed, and a measurement of % of initial allergen remaining was determined via the same MARIA testing 10 minutes after the addition of formulation into the chamber (each efficacy test identified as “Test” in FIG. 4).

The boxed section associated with each group of data points shows the standard deviation for each group, while the “+” associated with each group of data points represents the mean. Utilizing a suitable statistical analysis method, such as analysis of variance (ANOVA), confirms that introduction of the same formulation to each airborne allergen tested results in a statistically significant reduction, over a control, of active allergen remaining in the air and according to the results of a MARIA assay. An ELISA assay also would provide similar results. Thus, the data of this efficacy testing, as depicted in FIG. 4, shows that the effective amount of the droplets delivered into the air environment provides a statistically significant reduction, over a control, in one or more airborne allergens as measured by an enzyme linked immunoassay. Accordingly, the data provided in FIG. 4 demonstrates that the formulation provides a statistically significant reduction, over a control, in one or more airborne allergens as measured by an enzyme linked immunoassay.

Thus, anti-allergen formulations and delivery systems as described herein provide safe and effective neutralization of airborne allergens in enclosed environments such as any space or rooms in residential or commercial buildings.

In an embodiment, organic acids that are effective in neutralization of airborne allergens can be provided within the formulations that are recognized as safe in accordance with EPA and/or ECHA standards. An effective formulation can include one or a combination of organic acids to achieve a neutralization of airborne allergens in an air environment that is greater than about 50% after a select time period (e.g., within 15 minutes) of initial application or initial delivery of the formulation into the air environment.

For example, in this embodiment, an effective formulation can be delivered by a device in an effective amount to neutralize greater than about 50% of one or more airborne allergens present in an air environment (e.g., an enclosed air environment such as a room, office, hallway, closet, etc. and/or an enclosed air environment having a volume of at least about 1 m³) after a set time period (e.g., within 15 minutes) of initial application or initial delivery of the formulation into the air environment. Alternatively, in another embodiment, an effective formulation will have a statistically relevant reduction in one or more airborne allergens over the baseline or control (as shown, e.g., in FIG. 4).

Furthermore, effective formulations can comprise one or more organic acids and water, but being substantially free of any other ingredient or component other than the one or more organic acids and water. The effective formulations can further include one or more organic acids as active ingredients while being free of or substantially free of any other active ingredient, including but not limited to: oxidants, salts, silica materials, etc., that is also capable of neutralizing airborne allergens in an air environment.

While the invention has been described in detail and with reference to specific embodiments thereof, it will be apparent to one skilled in the art that various changes and modifications can be made therein without departing from the spirit and scope thereof.

What is claimed:

1. A system for neutralization of airborne allergens, the system comprising:
(a) a formulation comprising:
(i) an organic acid having a vapor pressure between about 0.001 Pa and about 172 Pa at room temperature and standard pressure and an aqueous solubility of at least about 24.99 grams organic acid per 100 grams water at room temperature and standard pressure;
(ii) a solvent selected from the group consisting of glycols, glycol ethers, glycol ether acetates and any combinations or mixtures thereof; and
(iii) optionally, one or more adjuvants selected from the group consisting of fragrances, waxes, dyes, colorants, stabilizers, thickeners, defoamers, hydrotropes, buffers, builders, enzymes, cloud point modifiers, preservatives, and any mixtures or combinations thereof; and
(b) a device that generates droplets of the formulation having a median particle size (Dv50) of no greater than about 120 micrometers;
wherein the system provides a statistically significant reduction, over a control, in one or more airborne allergens as measured by an enzyme linked immunoassay.

2. The system of claim 1, wherein the device generates droplets of the formulation having a median particle size (Dv50) of no greater than about 100 micrometers.

3. The system of claim 1, droplets of the formulation having a median particle size (Dv50) between 5 micrometers and 70 micrometers.

4. The system of claim 1, wherein the organic acid has a vapor pressure between about 0.4 Pa and about 172 Pa at room temperature and standard pressure.

5. The system of claim 1, wherein the organic acid is present in the formulation in an amount between about 0.2% and about 20% by weight of the formulation.

6. The system of claim 1, wherein the formulation comprises one or more organic acids selected from the group consisting of lactic acid, glycolic acid, butyric acid, pyruvic acid, maleic acid, and methane sulfonic acid.

7. The system of claim 1, wherein the formulation comprises lactic acid.

8. The system of claim 1, wherein the device delivers the formulation in an effective amount to neutralize greater than 50% of one or more airborne allergens present in an air environment within 15 minutes from initial delivery of the formulation into the air environment.

9. The system of claim 1, wherein the formulation further comprises one or more of a surfactant, a preservative, a buffer and a fragrance.

10. The system of claim 1, wherein the formulation has a pH between about 1 and about 6.5.

11. The system of claim 1, wherein the formulation comprises:
(a) lactic acid in an amount between about 0.2% and about 20% by weight of the formulation;
(b) a preservative in an amount between about 0.05% and about 0.2% by weight of the formulation;
(c) the solvent in an amount between about 1% and about 60% by weight of the formulation;
(d) a fragrance in an amount between about 0.25% and about 20% by weight of the formulation; and
(e) water.

12. The system of claim 1, wherein the formulation comprises:
(a) lactic acid in an amount between about 0.2% and about 20% by weight of the formulation;
(b) a preservative in an amount between about 0.05% and about 0.2% by weight of the formulation;
(c) a non-ionic surfactant in an amount between about 1% and about 5% by weight of the formulation selected from the group consisting of alkyl polyglucoside, alcohol ethoxylate, and any combinations or mixtures thereof;
(d) a fragrance in an amount between about 0.25% and about 0.4% by weight of the formulation; and
(e) water.

13. The system of claim 1, wherein the device comprises one or more of a pump sprayer, a mister, a nebulizer, a vaporizer, a cold fogger, a thermal fogger, an ultrasonic dispensing device, and an electrostatic sprayer.

14. A system for neutralization of airborne allergens, the system comprising:
(a) a formulation comprising an organic acid having a vapor pressure between about 0.001 Pa and about 172 Pa at room temperature and standard pressure and an aqueous solubility of at least about 24.99 grams organic acid per 100 grams water at room temperature and standard pressure; and
(b) a device that delivers the formulation as droplets and in an effective amount to neutralize greater than 50% of one or more airborne allergens present in an air environment.

15. The system of claim 14, wherein the device delivers the formulation as droplets and in an effective amount to neutralize greater than 50% of one or more airborne allergens present in the air environment within 15 minutes from initial delivery of the formulation into the air environment.

16. The system of claim 14, wherein the organic acid is present in the formulation in an amount between about 0.2% and about 20% by weight of the formulation.

17. The system of claim 14, wherein the organic acid has a vapor pressure between about 0.4 Pa and about 172 Pa at room temperature and standard pressure.

18. The system of claim 14, wherein the formulation comprises one or more organic acids selected from the group consisting of lactic acid, glycolic acid, butyric acid, pyruvic acid, maleic acid, and methane sulfonic acid.

19. The system of claim 14, wherein the formulation comprises lactic acid.

20. The system of claim 14, wherein the formulation comprises:
(a) lactic acid in an amount between about 0.2% and about 20% by weight of the formulation;
(b) a preservative in an amount between about 0.05% and about 0.2% by weight of the formulation;
(c) a non-ionic surfactant in an amount between about 1% and about 5% by weight of the formulation selected from the group consisting of: alkyl polyglucoside, alcohol ethoxylate, and any combinations or mixtures thereof;
(d) a fragrance in an amount between about 0.25% and about 0.4% by weight of the formulation; and
(e) water.

21. The system of claim 14, wherein the device comprises one or more of a pump sprayer, a mister, a nebulizer, a vaporizer, a cold fogger, a thermal fogger, an ultrasonic dispensing device, and an electrostatic sprayer.

22. A method of neutralizing airborne allergens within an air environment, the method comprising:
generating droplets of a formulation via a device and delivering an effective amount of the droplets into an air environment, one or more airborne allergens being present in the air environment;
wherein:
the formulation comprises water and an organic acid having a vapor pressure between about 0.001 Pa and about 172 Pa at room temperature and an aqueous solubility of at least about 24.99 grams organic acid per 100 grams water; and
the effective amount of the droplets delivered into the air environment provides a statistically significant reduction, over a control, in one or more airborne allergens as measured by an enzyme linked immunoassay.

23. The method of claim 22, wherein the droplets of formulation generated by the device have a median particle size (Dv50) of no greater than about 120 micrometers, and the formulation comprises one or more organic acids selected from the group consisting of lactic acid, glycolic acid, butyric acid, pyruvic acid, maleic acid, and methane sulfonic acid.
